(19) United States
(12) Patent Application Publication
(10) Pub. No.: US 2021/0260142 A1
(43) Pub. Date: Aug. 26, 2021

(54) COMPOSITIONS BASED ON DIETARY FIBERS AND ESSENTIAL OILS

(71) Applicant: Ralco Nutrition, Inc., Marshall, MN (US)

(72) Inventors: Richard Dale Lamb, Balaton, MN (US); Michael David Johnson, Balaton, MN (US); Robert Howard Stock, Blairstown, NJ (US)

(21) Appl. No.: 17/185,438

(22) Filed: Feb. 25, 2021

Related U.S. Application Data
(60) Provisional application No. 62/981,687, filed on Feb. 26, 2020.

(51) Int. Cl. A61K 36/15; A23K 50/75; A23K 20/163; A23K 50/80; A61P 31/04; A61K 47/44

(52) U.S. Cl. CPC A61K 36/15; A23K 50/75; A61K 47/44; A23K 50/80; A61P 31/04; A23K 20/163

(57) ABSTRACT

Embodiments of the present disclosure describe a composition comprising a dietary fiber, one or more essential oils, and a carrier. Embodiments of the present disclosure describe a method of preparing a composition comprising contacting a dietary fiber and one or more essential oils with a carrier sufficient to form a composition for enhancing immune responses in subjects having or susceptible to having infections and/or diseases caused by gastrointestinal pathogens. Embodiments of the present disclosure further describe a method of administering a composition comprising administering a composition to a subject having or susceptible to having an infection and/or disease caused by a pathogen, wherein the composition comprises a dietary fiber, one or more essential oils, and a carrier.

### BACKGROUND

Pathogenic infections and diseases in animals can have a significant impact on growth and productivity and thus can be the cause of substantial economic loss. Host protection against infections and diseases caused by infectious agents is provided by the immune system, a collection of molecular and cellular mechanisms and processes which function synergistically to either rid the host of the offending agents or control their proliferation. The immune system generates innate and adaptive immune responses that play an important role in protecting against infection and disease caused by pathogens. The innate immune responses of the immune system get involved early post-infection and generally includes those immune responses that occur rapidly after infection or development of disease. Their function is to control the extent of infection in an agent nonspecific manner They are initiated without prior sensitization to the pathogen, are not antigen specific, and are mediated directly by phagocytic cells such as macrophages, cytotoxic cells such as natural killer (NK) cells and antigen presenting cells such as dendritic cells (DCs), as well as indirectly by the cytokines produced by these cells. The innate immune response can also facilitate the development of adaptive immune responses.

The adaptive immune responses of the immune system is an acquired or adaptive immunity that is antigen specific and has a high degree of efficiency. The adaptive immune responses, however, take time to develop (e.g., 5-7 days) after initial infection or disease development and are thus slower than innate immune responses, which are rapid. The process of acquired immunity can involve an education of immune cells, resulting in the development of a highly specific, highly potent and long-lived response. This is mediated by cytotoxic T-lymphocytes (CTLs), helper T-lymphocytes and antibody-producing B-lymphocytes. Along these lines, adaptive immune responses are classified as either cellular (those mediated by CTLs) or humoral (antibody mediated responses), with helper T-lymphocytes facilitating both responses. Once developed, the adaptive immunity serves the clear the disease as well as to protect against its recurrence.

Despite both innate and adaptive immune responses, infections and diseases still cause damage, which can be in the form of, for example, reduced food efficiency, reduced body weight gain, reduced egg and milk production, etc. To combat this, hormones and antibiotics are commonly used. For example, antibiotics, such as coccidiostats, are often administered in sub-therapeutic doses to promote animal health and growth. However, sub-therapeutic antibiotics have been linked to pathogenic antibiotic resistance in animals, and their use as animal feed additives and growth promoters are banned in many countries. Similarly, animal hormonal supplements have been linked to detrimental side effects in animals as well as public health concerns for humans. Further, the need for alternatives to sub-therapeutic antibiotics and hormonal growth promoters is readily recognized by the significant economic incentive to market animal products as “natural,” “organic,” “hormone-free,” and “antibiotic-free.”

Accordingly, it would be desirable to provide a natural product as an alternative to hormones and/or antibiotics that minimizes damage to subjects by enhancing their immune responses against pathogens and other foreign invaders.

### SUMMARY

In general, embodiments of the present disclosure describe compositions, methods of preparing compositions, methods of preparing compositions, and the like. The compositions described herein can be used for enhancing immune responses in subjects having or susceptible to having infections and/or diseases caused by pathogens.

Embodiments of the present disclosure describe a composition comprising a dietary fiber, one or more essential oils, and a carrier.

Embodiments of the present disclosure describe a method of preparing a composition comprising contacting a dietary fiber and one or more essential oils with a carrier sufficient to form a composition for enhancing immune responses in subjects having or susceptible to having infections and/or diseases caused by gastrointestinal pathogens.

Embodiments of the present disclosure further describe a method of administering a composition comprising administering a composition to a subject having or susceptible to having an infection and/or disease caused by a pathogen, wherein the composition comprises a dietary fiber, one or more essential oils, and a carrier.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method 100 of preparing a composition, according to one or more embodiments of the present disclosure.

FIG. 2, illustrates a block flow diagram of a method 200 of making an essential oil composition for use in the method 100, according to one or more embodiments of the present disclosure.

FIG. 3 is a flowchart of a method 300 of administering a composition, according to one or more embodiments of the present disclosure.

### DETAILED DESCRIPTION

The present disclosure relates to compositions based on select combinations of dietary fibers and essential oils for enhancing immune responses against pathogens and other foreign invaders. In particular, the compositions described herein can enhance the innate immune responses and adaptive immune responses of subjects and can be administered to treat or prevent infection and disease. While not wishing to be bound to a theory, it is believed that the administration of the compositions described herein enhance immune responses in subjects by boosting immunoglobulin production, activating natural killer (NK) cells, and/or mobilizing lymphocytes (e.g., gamma delta T cells and CD4 T cells), among other things. For example, the immunoglobulin production and activation of NK cells can effectively reduce pathogen levels (e.g., destroy and/or inhibit the growth of pathogens) and minimize damage therefrom, while the mobilization of the gamma delta T cells can stimulate the process through which the subject acquires immunity. There is thus a surprising synergy between the dietary fibers and essential oils that can be used to treat or prevent infection and disease.

The compositions described herein can be particularly effective against gastrointestinal pathogens (e.g., enteric diseases, etc.) and in improving mucosal immunity. One non-limiting example of a disease against which the compositions described herein are particularly effective is coccidiosis, which is a parasitic disease of the intestinal tract. It has been discovered that the administration of the compositions to subjects having coccidiosis not only minimizes damage caused by the parasite to the intestinal mucosal barrier and provides control over (e.g., reduce and/or maintain) their presence in the gastrointestinal tract, but also assists in the process through which the subject acquires immunity to the coccidia. While not wishing to be bound to a theory, it is believed that the compositions can enhance a subject's innate immune response to coccidia by increasing production of immunoglobulin A (IgA), including secretary IgA or SIgA, which protects the intestinal epithelium from enteric toxins and pathogens. This synergistically minimizes damage to the subject while its adaptive immune response sends gamma delta T cells to the intestinal tract to initiate the slower process through which the subject acquires immunity. The result is healthier subjects and reduced or no occurrence of mortality, among other things.

The compositions described herein can also enhance immune responses against pathogens other than coccidia. For example, the pathogens can include microorganisms, such as a virus, bacterium, protozoan, prion, viroid, or fungus, as well as parasites, such as worms, insect larvae, and other types of small animals that can produce disease. In some embodiments, the pathogens can be selected from Eimeria tenella, Eimeria maxima, Eimeria acervulina, Escherichia E. coli, Salmonella typhimurium, Salmonella choleraesuis, Salmonella pullorum, Pseudomonas aeruginosa, Vibrio parahaemolyticus, Vibrio, Aeromonas, Yersinia entercolitica, Klebsiella pneumoniae, Clostridium perfringens, Staphylococcus aureus, Streptococcus agalactiae, Streptococcus uberis, Listeria monocytogenes, Bacillus cereus, Myobacterium avium, Aspergillus, Penicillium, Candida albicans, Streptococcus suis, Salmonella enteritidis, Campylobacter jejuni, Campylobacter coli, and combinations thereof. These shall not be limiting, as the compositions can enhance immune responses in subjects having infection and/or disease caused by other types of species or genus of pathogens.

### Definitions

The terms recited below have been defined as described below. All other terms and phrases in this disclosure shall be construed according to their ordinary meaning as understood by one of skill in the art.

As used herein, “subject” refers to any organism that can benefit from an improved innate and/or adaptive immune response. Non-limiting examples of “subjects” can include human and non-human subjects, including, but not limited to, avian, bovine, canine, equine, feline, hircine, murine, ovine, and porcine animals, such as, poultry, swine, beef cattle, horses, dogs, cats, goals, etc. As used herein, “poultry” refers to birds of the order Galliformes including ordinary domestic fowl or chicken (Gallus domesticus), turkeys (Meleagris), pheasants (Phasianus), partridges (Perdix), grouse (Lagopus), guinea fowl (Numida) peacocks (Pavo), and also birds of the order Anseriformes such as ducks (Anas) and geese (Anser).

As used herein, “pathogen” refers to any species capable of producing infection and/or disease. The pathogens can include microorganisms, such as a virus, bacterium, protozoan, prion, viroid, or fungus, as well as parasites, such as worms, insect larvae, and other types of small animals that can produce disease. Examples of pathogens include, but are not limited to, Eimeria tenella, Eimeria maxima, Eimeria acervulina, Escherichia E. coli, Salmonella typhimurium, Salmonella choleraesuis, Salmonella pullorum, Pseudomonas aeruginosa, Vibrio parahaemolyticus, Vibrio, Aeromonas, Yersinia entercolitica, Klebsiella pneumoniae, Clostridium perfringens, Staphylococcus aureus, Streptococcus agalactiae, Streptococcus uberis, Listeria monocytogenes, Bacillus cereus, Myobacterium avium, Aspergillus, Penicillium, Candida albicans, Streptococcus suis, Salmonella enteritidis, Campylobacter jejuni, Campylobacter coli, and combinations thereof.

As used herein, “enhancing” in the context of immune responses refers to one or more of enhancing, priming, potentiating, activating, stimulating, augmenting, boosting, and/or amplifying immune responses in subjects.

As used herein, “immune response” refers to any action relating to a host's immune system and can include, for example, an innate immune response and an adaptive immune response. As used herein, the term “innate immune response” can refer to a nonspecific immune response. As used herein, the term “adaptive immune response” can refer to a specific immune response.

As used herein, “plants” and “plant derivatives” can refer to any portion of a growing plant, including the roots, stems, stalks, leaves, branches, seeds, flowers, fruits, and the like. For example, cinnamon essential oil can be derived from the leaves or bark of a cinnamon plant.

As used herein, the term “essential oils” refers to aromatic, volatile liquids extracted from plant material. Essential oils are often concentrated hydrophobic liquids containing volatile aroma compounds. Essential oil chemical constituents can fall within general classes, such as terpenes (e.g., p-Cymene, limonene, sabinene, a-pinene, y-terpinene, b-caryophyllene), terpenoids (e.g., citronellal, thymol, carvacrol, carvone, borneol) and phenylpropanoids (e.g., cinnamaldehyde, eugenol, vanillin, safrole). Essential oils can be natural (i.e., derived from plants), or synthetic.

As used herein “cinnamon essential oil” refers to one or more of natural cinnamon oil (i.e., essential oil derived from plants in the Cinnamomum genus), or synthetic cinnamon oil. Synthetic cinnamon essential oil can comprise synthetic cinnamaldehyde. Synthetic cinnamon essential oil can further comprise one or more major constituents of natural cinnamon essential oil. A major constituent is one which comprises at least 1 wt. %, at least 2.5 wt. %, or at least 5 wt. % of a natural essential oil assay.

As used herein “thyme essential oil” refers to one or more of natural thyme oil (i.e., essential oil derived from plants in the Thymus genus), or synthetic thyme oil. Synthetic thyme essential oil can comprise synthetic thymol. Synthetic thyme essential oil can further comprise one or more major constituents of natural thyme essential oil.

As used herein “oregano essential oil” refers to refers to one or more of natural oregano oil (i.e., essential oil derived from plants in the Origanum genus), or synthetic oregano oil. Synthetic oregano essential oil can comprise synthetic carvacrol. Synthetic oregano essential oil can further comprise one or more major constituents of natural oregano essential oil.

As used herein, the term “emulsion” refers to a fine dispersion of droplets of one liquid in which the liquid is not substantially soluble or miscible. An essential oil may be emulsified or substantially emulsified within an aqueous carrier, for example.

As used herein, the term “emulsifier” refers to a substance that stabilizes an emulsion. The emulsifier can utilize physical properties, chemical properties, or utilize both physical and chemical properties to interact with one or more substances of an emulsion. Tannic acid is an example of an emulsifier for essential oils and water.

As used herein, the term “tannin compound” refers to a polyphenolic biomolecule including at least twelve hydroxyl groups and at least five phenyl groups. Tannin compounds include compounds utilizing gallic acid, flavone and phloroglucinol as base units. Tannic acid (C₇₆H₅₂O₄₆) is one form of a tannin compound. Tannic acid can include quercitannic acid and gallotannic acid, for example.

As used herein, “carrier” refers to a substance that physically binds or combines, or chemically binds or combines, with a target or active substance to facilitate the use, storage, or application of the target or active substance. Carriers are often inert materials, but can also include non-inert materials when compatible with the target or active substances. Examples of carriers include, but are not limited to, water for compositions that benefit from a liquid carrier, or diatomaceous earth for compositions that benefit from a solid carrier.

As used herein, “enzymes” refers to one or more biological molecules capable of breaking down cellulosic material. Enzymes include starch, proteins, non-starch polysaccharides, both soluble and insoluble, lignins and those biological molecules that facilitate chemical reactions within plants and animals.

### Compositions for Enhancing Immune Responses

Embodiments of the present disclosure describe compositions comprising at least a dietary fiber, essential oil, and carrier. The compositions are based on select combinations of dietary fibers and essential oils that enhance immune responses in subjects having or susceptible to having an infection and/or disease caused by a pathogen. The compositions can be provided in liquid or solid form. For example, in one embodiment, the compositions comprise a dietary fiber dissolved in a liquid carrier and one or more essential oils emulsified, dispersed, or suspended in the liquid carrier. In another embodiment, the compositions comprise a dietary fiber, one or more essential oils, and a solid carrier, wherein the dietary fiber and the essential oils are absorbed, and in some instances adsorbed, by the solid carrier. These are only provided as examples of the forms in which the compositions can be provided and thus shall not be limiting, as the compositions described herein can be provided in a variety of other forms without departing from the scope of the present disclosure.

The ratio of dietary fibers to essential oils in the composition can range from about 0:100 to about 100:0, or any increment thereof. In an embodiment, the ratio of dietary fibers to essential oils in the composition can be about 10:1 to about 20:1. For example, the ratio of dietary fibers to essential oils can be about 10:1, about 11:1, about 12:1, about 13:1, about 14:1 about 15:1, about 16:1, about 17:1, about 18:1, about 19:1, about 20:1, or any increment thereof. In one embodiment, the ratio of dietary fibers to essential oils in the composition is about 13:1.

The amount of dietary fibers present in the compositions can be based on the body weight or mass of the subject to which the composition is being administered. In an embodiment, the amount of dietary fiber present in the compositions can range from about 1 mg of dietary fiber per kg of body weight of the subject to about 50 mg of dietary fiber per kg of body weight of the subject, or any increment thereof. For example, the amount of dietary fiber present in the composition can be about 1 mg of dietary fiber per kg of body weight, about 10 mg of dietary fiber per kg of body weight, about 20 mg of dietary fiber per kg of body weight, about 30 mg of dietary fiber per kg of body weight, about 40 mg of dietary fiber per kg of body weight, or about 50 mg of dietary fiber per kg of body weight, among others. In an embodiment, the amount of dietary fiber present in the composition can range from about 10 mg of dietary fiber per kg of body to about 20 mg of dietary fiber per kg of body weight.

The amount of essential oils present in the compositions can also be based on the body weight or mass of the subject to which the composition is being administered. In an embodiment, the total amount of essential oils present in the compositions can range from about 0.1 mg to 1000 mg of essential oil (e.g., pure essential oil, or excluding species other than the essential oil) per kg of body weight of the subject, or any increment thereof. The amount of essential oil per kg of body weight can depend on the essential oil, its minimum inhibitory concentration (MIC), and the pathogen. For example, essential oils, such as cinnamon essential oil, thyme essential oil, and/or oregano essential oil, among others, can exhibit low MICs with respect to a plurality of pathogens. In an embodiment, the amount of essential oil suitable for enhancing immune responses in subjects can range from about 0.5 mg to about 1.0 mg of essential oil per kg of body weight. Other essential oils may require more than 1.0 mg of essential oil per kg of body weight in order to enhance immune responses in subjects.

### Dietary Fibers

The dietary fibers can be selected from larch arabinogalactan, arabinogalactan, polydextrose, chitin, psyllium, methylcellulose, hydrolyzed guar, guar, soy polysaccharide, oat bran, pectin, inulin, fructooligosaccharides (FOS), gum arabic, and combinations thereof. These dietary fibers can offer various advantageous attributes including, but not limited to, one or more of being a natural fiber source, an Association of Official Analytical Chemists (AOAC) test fiber method, being a soluble or highly soluble fiber (e.g., water-soluble), having a low sensory impact, exhibiting pH and/or temperature stability, having hypoallergenicity, not requiring label warnings, having low or no flatulation, functioning as a bulking agent, slowing transit time, lowering stool pH, lowering cholesterol, increasing the ratio of HDL:LDL, pre-adapting GI tracts, being fermented completely and/or slowly, producing short-chain fatty acids, generating butyric acid, reducing glycermic index, reducing insulin response, promoting Bifidobacteria, promoting Lactobacillus, promoting growth factors, creating ideal growth, activating lymphocytes, activating macrophage, stimulating interferon, stimulating interleukin, and activating natural killer (NK) cells. These attributes can serve as a basis for selecting dietary fibers.

In one embodiment, the dietary fiber can be selected to be larch arabinogalactan. The larch arabinogalactan can generally include any composition comprising arabinogalactan and optionally other species, such as polyphenols. The larch arabinogalactan can be extracted or derived from any species in the genus Larix. For example, species of the genus Larix include, but are not limited to, Larix laricina, Larix lyallii, Larix leptolepis, Larix occidentalis, Larix decidua, Larix dahurica, Larix sibirica, Larix gmelinii, Larix kaempferi, Larix czekanowskii, Larix potaninii, Larix mastersiana, Larix griffithii, and hybrids thereof. The larch arabinogalactan is available from commercial sources. It can be provided in solid form, such as in the form of a powder, or it can be provided in liquid form.

The arabinogalactan can be characterized as a water-soluble, highly or densely branched polysaccharide. The arabinogalactan can generally include any compound composed of galactose units and arabinose units in an approximate ratio of about 100:1 to about 1:1. For example, the arabinogalactan can have a galactan backbone with side chains containing galactose units and arabinose units, wherein a ratio of the galactose units to arabinose units is about 6:1 or about 7.5:1. In an embodiment, the arabinogalactan can be characterized as having a backbone of (1→3)-linked β-D-galactopyranosyl units, each of which can bear a substituent at the C6 position. Most of these side chains can be galactobiosyl units containing a (1→6)-β-D-linkage and α-L-arabinofuranosyl units. These shall not be limiting, as the arabinogalactan can also include arabinogalactan derivatives, such as lipidated and/or quaternized forms of arabinogalactan.

The arabinogalactan can vary in molecular weight from low molecular weight polymers to large macromolecules. The molecular weight of the arabinogalactan can range from about 1,000 Daltons to about 2,500,000 Daltons, or any increment thereof. For example, the molecular weight of the arabinogalactan can range from about 6,000 Daltons to about 2,500,000 Daltons, about 6,000 Daltons to about 300,000 Daltons, about 3,000 Dalton to about 120,000 Dalton, about 15,000 Dalton to about 60,000 Dalton, or about 40,000 Dalton to about 60,000 Dalton, among other ranges.

The larch arabinogalactan can include other species. For example, typically, the larch arabinogalactan comprises polyphenols. The polyphenols can include any compound having two or more phenol groups or moieties. Examples of polyphenols include, but are not limited to, one or more of flavonoids, aromadendrines, anthocyanins, catecholins, catechins, and taxifolins. In an embodiment, the polyphenols include at least flavonoids, such as quercetin. The larch arabinogalactan typically comprises about 1 wt % to about 4 wt % of polyphenols; however, higher and lower concentrations are possible and within the scope of the present disclosure.

The larch arabinogalactan can be selected to, among other things, inhibit the growth of pathogens (e.g., pathogen growth can be inhibited in the presence of polyphenols); increase the production of short chain fatty acids (e.g., butyrate, propionate, acetate, etc.); preferentially promote the growth of beneficial bacteria (e.g., Bifidobacteria, Lactobacillus, etc.) and by that reduce the presence of harmful pathogens; inhibit pathogen attachment to the epithelial wall; decrease clostridia; boost or increase immunoglobulin production (e.g., IgA and/or SIga) to initiate inflammatory reactions, trigger respiratory burst activity by polymorphonuclear leukocytes, as well as result in cell mediated cytotoxicity, degranulation of eosinophils/basophils, phagocytosis by monocytes, macrophages, neutrophils, and eosinophils; stimulate B plasma cells; activate NK cells; minimize damage to the gastrointestinal tract (e.g., intestinal mucosal barrier); stimulate healthy macrophage increase; enhance NK cell cytotoxicity against K562 tumor cells through IFN gamma production; increase TNF alpha IL-1 and -6; increase circulating white blood cell counts; increase circulating neutrophils; increase circulating monocytes; improve gut health; reduce fecal ammonia and dry digestive matter; reduce diarrhea index; modulate glucose and insulin levels; promote lean build and weight gain; provide a natural source of antioxidants (e.g., quercetin); reduce illness risk; reduce incidence of scours; and/or lower toxicity, odor, and soften fecal matter.

### Essential Oils

The compositions can further comprise one or more essential oils. As used herein, “essential oils,” “essential oil compositions,” and other similar variations can be used interchangeably. In an embodiment, the essential oils can be selected from oregano essential oil, thyme essential oil, cinnamon bark essential oil, lavender essential oil, bay leaf essential oil, cinnamon leaf essential oil, laurel leaf essential oil, lemon grass essential oil, spearmint essential oil, peppermint essential oil, rosemary essential oil, clary sage essential oil, and combinations thereof. In an embodiment, the one or more essential oil compositions can be present as an emulsion, wherein the average particle size of the one or more essential oils in the emulsion is less than or about 25 microns.

In general, the essential oil compositions as provided herein contain essential oils derived from plants (i.e., “natural” essential oils) and additionally or alternatively their synthetic analogues. Many embodiments comprise a combination of essential oils. Some embodiments comprise a combination of natural and synthetic essential oils. In some embodiments, synthetic essential oils can be a “nature's equivalent” synthetic blend, which generally mimics an essential oil assay of a natural essential oil by including at least 5, at least 10, at least 15, or at least 20 of the most critical essential oils within a natural essential oil. A critical essential oil can be determined by weight percent, and/or by pharmacological efficacy. For example, a nature's equivalent synthetic oil can comprise the following constitutions as provided in Table 1:

TABLE 1
Nature's Equivalent Synthetic Thyme Essential Oil:

| Constituent | Wt. % |
|---|---|
| Thymol | 42.7-44.08 |
| para-Cymene | 26.88-27.09 |
| Linalool | 4.3-4.34 |
| alpha-Pinene | 4.1-4.26 |
| alpha-Terpineol | 3.14-3.14 |
| 1,8-Cineole | 2.82-3.01 |
| beta-Caryophellene | 1.98-2.27 |
| Limonene | 1.59-1.78 |
| delta-3-Carene | 1.3-1.41 |
| beta-Myrcene | 1.26-1.31 |
| Linalyl Acetate | 1.11-1.24 |
| beta-Pinene | 1.04-1.22 |
| Terpinen-4-ol | 0.96-1.14 |
| alpha-Caryophyllene | 0.71-0.71 |
| gamma-Terpinene | 0.7-0.7 |
| Sabinene | 0.37-0.5 |
| Borneol | 0.27-0.32 |
| Camphene | 0.13-0.17 |

The disclosure herein indicates the efficacy of compositions comprising a plurality of essential oils which provide a synergistic effect beyond essential oils utilized in isolation. Further, essential oil compositions provided herein do not exhibit antagonistic effect between essential oil moieties within a composition. An essential oil composition generally includes an essential oil fraction and one or more additional components. The ratio of the essential oil fraction to the one or more additional components in a composition can depend on several factors such as administration method, and the nutritional/health needs and/or palate of a consuming subject, among others. In many embodiments, a consuming subject comprises an animal Compositions can comprise additional components including carriers, emulsifiers, and stabilizers, among others. Compositions as provided herein can be in the form of an emulsion.

The essential oils present in some embodiments can include oils from the classes of terpenes, terpenoids, phenylpropenes and combinations thereof. The essential oils present in some embodiments can include oils of plants from the Labiatae or Lamiaceae family, and the Lauraceae family, including hybrids of plants from one or both families. Suitable essential oils from the Lauraceae family can comprise those from the Cinnamomum genus. Within the Cinnamomum genus, suitable species can include Cinnamomum burmannii, Cinnamomum cassia, Cinnamomum camphora, Cinnamomum loureiroi, Cinnamomum mercadoi, Cinnamomum oliveri, Cinnamomum osmophloeum, Cinnamomum ovalifolium, Cinnamomum parthenoxylon, Cinnamomum pedunculatum, Cinnamomum subavenium, Cinnamomum tamala, Cinnamomum verum, Cinnamomum verum, and hybrids thereof.

Suitable essential oils from the Lamiaceae family can comprise those from one or more of the Thymus genus, the Origanum genus, the Monarda genus. Within the Thymus genus, a non-limiting list of suitable species can include Thymus caespititius, Thymus capitatus, Thymus carnosus, Thymus citriodorus, Thymus glandulosus, Thymus Herba-borana, Thymus hyemalis, Thymus integer, Thymus pseudolanuginosus (formerly T. lanuginosus), Thymus mastichinia, Thymus montanus, Thymus moroderi, Thymus pannonicus, Thymus praecox, Thymus pulegioides, Thymus serpyllum, Thymus vulgaris, Thymus zygis, and hybrids thereof. Within the Origanum genus, a non-limiting list of suitable species can include Origanum amanum, Origanum compactum, cordifolium, Origanum dictamnus, Origanum laevigatum, Origanum libanoticum, Origanum majorana, Origanum microphyllum, Origanum onites, Origanum rotundifolium, Origanum scabrum, Origanum syriacum, Origanum vulgare, and hybrids thereof. Within the Monarda genus, a non-limiting list of suitable species can include Monarda citriodora, Monarda clinopodioides, Monarda didyma, Monarda fistulosa, Monarda media, Monarda punctata, and hybrids thereof.

The essential oils present in some embodiments can further include lavender essential oils from the Lavandula genus, Mexican bay leaf essential oils from the Liteas genus (e.g., L. glaucescens), West Indian bay tree essential oils from the Pimenta genus (e.g., P. racemosa), Indonesian bay leaf essential oils from the Syzygium genus, bay laurel essential oils from the Laurus genus (e.g., L. nobilis), California bay laurel essential oils from the Umbellularia genus (e.g., U. californica), lemon grass essential oils from the Cymbopogon genus (e.g., C. ambiguous, C. citratus, C. flexuosus, C. martini, C. nardus, C. schoenanthus), spearmint and peppermint essential oils from the Mentha genus (e.g., M. spicata, M. piperita), rosemary essential oils from the Rosmarinus genus (e.g., R. officinalis), sage essential oils from the Salvia genus (e.g., S. sclarea), hybrids thereof, and combinations thereof.

In some embodiments, an essential oil composition can include an essential oil fraction comprising three essential oils from the Lauraceae family and/or the Lamiaceae family. In some embodiments, an essential oil composition can include an essential oil fraction comprising cinnamon essential oil from the Cinnamomum genus, thyme essential oil from the Thymus genus, and oregano essential oil the Origanum genus. In some embodiments, an essential oil composition can include an essential oil fraction comprising synthetic cinnamaldehyde and thyme essential oils from the Thymus genus and oregano essential oil from the Origanum genus. In some embodiments, oregano essential oil can comprise carvacrol. Additionally or alternatively, thyme essential oil can comprise thymol.

In some embodiments, the essential oil fraction can comprise about 1% to about 49.5% oregano essential oil, about 1% to about 49.5% thyme essential oil, and about 1% to about 49.5% cinnamon essential oil. In other embodiments, the essential oil fraction can comprise about 5% to about 47.5% oregano essential oil, about 5% to about 47.5% thyme essential oil, and about 5% to about 47.5% cinnamon essential oil. In other embodiments, the essential oil fraction can comprise about 10% to about 45% oregano essential oil, about 10% to about 45% thyme essential oil, and about 10% to about 45% cinnamon essential oil. In other embodiments, the essential oil fraction can comprise about 15% to about 42.5% oregano essential oil, about 15% to about 42.5% thyme essential oil, and about 15% to about 42.5% cinnamon essential oil. In other embodiments, the essential oil fraction can comprise about 20% to about 40% oregano essential oil, about 20% to about 40% thyme essential oil, and about 20% to about 40% cinnamon essential oil. In other embodiments, the essential oil fraction can comprise about 25% to about 37.5% oregano essential oil, about 25% to about 37.5% thyme essential oil, and about 25% to about 37.5% cinnamon essential oil. In other embodiments, the essential oil fraction can comprise about 30% to about 35% oregano essential oil, about 30% to about 35% thyme essential oil, and about 30% to about 35% cinnamon essential oil. In other embodiments, the essential oil fraction can comprise about 33.33% oregano essential oil, about 33.33% thyme essential oil, and about 33.33% cinnamon essential oil.

Many essential oil compositions comprise an essential oil fraction comprising an effective amount of carvacrol, an effective amount of thymol, and an effective amount of cinnamaldehyde. In an essential oil composition including an essential oil fraction comprising oregano essential oil, thyme essential oil, and cinnamon essential oil, the essential oil fraction can comprise three or more natural essential oils wherein the combined essential oils comprise at least an effective amount of carvacrol, at least an effective amount of thymol, and at least an effective amount of cinnamaldehyde. Suitable essential oils can include essential oils from the Cinnamomum genus, essential oils from the Origanum genus, essential oils from the Thymus genus, essential oils from the Monarda genus (e.g., M. citriodora, M. clinopodioides, M. didyma, M. fistulosa, M. media, M. punctata), essential oils from the Trachyspermum genus (e.g., T. ammi), essential oils from the Nigella genus (e.g., N. sativa), and combinations thereof. Other essential oils can be used such that effective amounts of carvacrol, thymol, and cinnamaldehyde are achieved in the essential oil fraction.

In an essential oil composition including an essential oil fraction comprising oregano essential oil, thyme essential oil, and synthetic cinnamaldehyde, the essential oil fraction can comprise two or more natural essential oils and synthetic cinnamaldehyde, wherein the combined essential oils and synthetic cinnamaldehyde comprise at an effective amount of carvacrol, at least an effective amount of thymol, and at least an effective amount of cinnamaldehyde. Suitable essential oils can include essential oils from the Cinnamomum genus, essential oils from the Origanum genus, essential oils from the Thymus genus, essential oils from the Monarda genus (e.g., M. didyma, and M. fistulosa), essential oils from the Trachyspermum genus (e.g., T. ammi), essential oils from the Nigella genus (e.g., N. sativa), and combinations thereof. Still other natural essential oils can be used such that effective amounts of carvacrol, thymol, and cinnamaldehyde are achieved in the essential oil fraction.

Some essential oil compositions comprise an essential oil fraction comprising an effective amount of carvacrol, an effective amount of thymol, and an effective amount of cinnamaldehyde. An effective amount of carvacrol can comprise at least about 10 wt. %, at least about 15 wt. %, at least about 20 wt. %, at least about 25 wt. %, at least about 30 wt. %, or at least about 33 wt. % of the essential oil fraction. An effective amount of thymol can comprise at least about 10 wt. %, at least about 15 wt. %, at least about 20 wt. %, at least about 25 wt. %, at least about 30 wt. %, or at least about 33 wt. % of the essential oil fraction. An effective amount of cinnamaldehyde can comprise at least about 10 wt. %, at least about 15 wt. %, at least about 20 wt. %, at least about 25 wt. %, at least about 30 wt. %, or at least about 33 wt. % of the essential oil fraction. In some embodiments, oregano essential oil can be replaced by one or more oils which include at least 45 wt. % carvacrol, at least 55 wt. % carvacrol, at least 65 wt. % carvacrol, or at least 75 wt. % carvacrol. In some embodiments, thyme essential oil can be replaced by one or more oils which include at least 30 wt. % thymol, at least 35 wt. % thymol, at least 40 wt. % thymol, or at least 45 wt. % thymol. In some embodiments, cinnamon essential oil can be replaced by one or more oils which include at least 35 wt. % cinnamaldehyde, at least 40 wt. % cinnamaldehyde, at least 50 wt. % cinnamaldehyde, or at least 75 wt. % cinnamaldehyde. Suitable sources of effective amounts of carvacrol, thymol, and/or cinnamaldehyde can include natural essential oils and/or synthetic essential oils.

Essential oil compositions can further comprise one or more of an effective amount of paracymene, an effective amount of eugenol, or an effective amount of citronella. An effective amount of paracymene can comprise at least about 5 wt. %, at least about 7.5 wt. %, at least about 10 wt. %, or at least about 12.5 wt. % of the essential oil fraction. An effective amount of eugenol can comprise at least about 5 wt. %, at least about 7.5 wt. %, at least about 10 wt. %, or at least about 12.5 wt. % of the essential oil fraction. An effective amount of citronella can comprise at least about 5 wt. %, at least about 7.5 wt. %, at least about 10 wt. %, or at least about 12.5 wt. % of the essential oil fraction.

In some embodiments, the essential oil fraction comprises 100% of the essential oil composition. An essential oil composition can further comprise a carrier. Carriers are ideally inert materials which do not react with the active components (i.e., the essential oil fraction) of the composition chemically, or bind the active components physically by adsorption or absorption. Liquid carriers include water, pure water, such as reverse osmosis water, milk, milk replacers, natural and/or commercial liquid feeds, or other liquids germane to animal dietary needs. Milk replacers can be formulated to generally mimic the content of milk. For example, a milk replacer can have a composition similar to that shown in Table 2:

TABLE 2
Example Milk Replacer Formula

| | |
|---|---|
| Crude Protein, minimum | 22.50% |
| Lysine, minimum | 1.60% |
| Crude Fat, minimum | 16.50% |
| Crude Fiber, maximum | 0.50% |
| Calcium, minimum | 0.65% |
| Calcium, maximum | 1.15% |
| Phosphorus, minimum | 0.60% |
| Sodium, minimum | 0.50% |
| Sodium, maximum | 1.00% |
| Selenium, minimum | 0.25 ppm |
| Zinc, minimum | 50 ppm |

The composition can be at least about 50% liquid carrier by weight, at least about 75% liquid carrier by weight, at least about 85% liquid carrier by weight, or at least about 90% liquid carrier. In some embodiments, the composition will be about 80% to about 99% liquid carrier, about 85% to about 98% liquid carrier, about 90% to about 95% liquid carrier, or about 91% to about 94% liquid carrier. In other embodiments, the composition can be about 60% liquid carrier to about 74% liquid carrier, about 63% liquid carrier to about 71% liquid carrier, about 66% liquid carrier to about 68% liquid carrier, or about 67% liquid carrier.

Solid carriers can include limestone, diatomaceous earth, and animal feed. Carriers such as limestone, diatomaceous earth, and the like, are useful pre-feed carriers in that they may be first combined with an essential oil fraction to facilitate transportation and/or subsequent combination of the essential oil composition with a dry carrier such as animal feed Animal feed can include hay, straw, corn husks, wheat, oats, barley, seeds, commercial livestock feed, and the like. In some embodiments where an essential oil composition comprises an essential oil fraction and a pre-feed carrier, the ratio of pre-feed carrier to the essential oil fraction can be at least 10:1, at least 15:1, at least 17:1, at least 18:1, or at least 20:1. In some embodiments, where an essential oil composition comprises an essential oil fraction and a carrier, with or without a pre-feed carrier, the ratio of carrier to the essential oil fraction can be at least about 1,000:1, at least about 4,500:1, at least about 9,000:1, at least about 20,000:1, at least about 35,000:1, or at least about 50,000:1.

The total amount of carrier in a composition can be determined based on the dietary needs of an animal, the tolerance of an animal to essential oil fraction, and other factors. Tolerance can include one or more of an animal's palatability and gastrointestinal tolerance to an essential oil fraction.

An essential oil composition can further comprise one or more emulsifiers. An emulsified essential oil fraction can increase the efficacy of an essential oil composition when ingested by a subject, and can make essential oil compositions more palatable to animals which consume the compositions orally. An essential oil fraction can be combined with an emulsifier and a dry carrier, or alternatively an essential oil fraction can be combined with an emulsifier and a liquid carrier, as disclosed above, to form an emulsion. The emulsifier can be combined with an essential oil fraction in a ratio of about 3:1 to about 1:3, about 2:1 to about 1:2, about 1.5:1 to about 1:1.5, or about 1:1. An essential oil composition comprising an essential oil fraction, a liquid carrier, and an emulsifier can have an average essential oil droplet size of less than about 25 microns, less than about 15 microns, less than about 10 microns, less than about 7.5 microns, or less than about 5 microns. In some embodiments, the average droplet size is less than about 7 microns, less than about 6 microns, less than about 5 microns, less than about 4 microns, or less than about 3 microns. As used herein, “droplet size” refers to the average size of an essential oil droplet within an emulsion.

An emulsifier combined with a liquid carrier can generally be referred to as a liquid emulsifier. In some embodiments, an emulsion can comprise up to about 35%, up to about 40%, up to about 45%, or up to about 50% essential oil fraction and emulsifier, with the balance comprising a liquid carrier. In some embodiments, an emulsion can comprise less than about 20%, less than about 15%, less than about 10%, about 5%, or less than about 5% essential oil fraction and emulsifier, with the balance comprising a liquid carrier. In some embodiments, an emulsion can comprise about 40% to about 60%, or about 45% to about 55% essential oil fraction and emulsifier, with the balance comprising a liquid carrier. In some embodiments, an emulsion can comprise about 1% to about 10%, about 2.5% to about 7.5%, or about 5% essential oil fraction and emulsifier, with the balance comprising a liquid carrier. In many embodiments the liquid carrier is water. The liquid carrier content can vary depending on the amount and type of emulsifier.

One or more emulsifiers can be used to form an emulsion. In some embodiments, one or more emulsifiers can additionally or alternatively be used as a stabilizer. Stabilizers can be used to alter the viscosity of an emulsion. Altering a viscosity can include maintaining a viscosity, increasing a viscosity, or decreasing a viscosity. A suitable emulsifier can be an emulsifier capable of achieving a threshold droplet size. In some embodiments a suitable emulsifier can achieve a suitable emulsion droplet size of less than about 25 microns, less than about 15 microns, less than about 10 microns, less than about 7.5 microns, or less than about 5 microns. In other embodiments, a suitable emulsifier can achieve a suitable emulsion droplet size of less than about 7 microns, less than about 6 microns, less than about 5 microns, less than about 4 microns, or less than about 3 microns. An emulsion having a droplet size below a suitable threshold enhances the efficacy of an essential oil composition.

A suitable emulsifier is larch arabinogalactan. Arabinogalactan generally comprises arabinose and galactose monosaccharides, and can be synthetic or natural. Natural arabinogalactan can be derived from plants or microbes. For example, arabinogalactan can be derived from larch trees, and many fruits, vegetables, and beans. In some embodiments, arabinogalactan is a preferred emulsifier because it is capable of achieving a desired droplet size and also acts as an antioxidant against many ROS, including peroxyl radicals, hydroxyl radicals, peroxynitrite, superoxide anions, and singlet oxygen. Accordingly, the hydrophilic characteristics of arabinogalactan enhance the cellular coverage of an essential oil composition. A particular type of arabinogalactan is larch arabinogalactan. Other suitable emulsifiers include polydextrose, chitin, psyllium, methyl-cellulose, hydrolyzed guar, guar, soy polysaccharide, oat bran, pectin, inulin, Fructooligosaccharides (FOS), xanthan gum, alginate, chemically modified cellulosic, Acacia, and gum Arabic.

In some embodiments, a suitable emulsifier can include a tannin compound, such as tannic acid. Tannin can be used as an alternative to or in combination with the emulsifiers described above. In some embodiments, a liquid emulsifier can comprise about 100% tannic acid, about 80% to about 95% tannic acid, about 60% to about 85% tannic acid, about 40% to about 60% or about 1% to about 50% tannic acid, with the balance being a liquid carrier.

An essential oil composition can further comprise one or more dedusting agents. Dedusting agents can comprise vegetable oil, olive oil, mineral oil and the like. The amount of dedusting agent in an essential oil composition can be determined based on the amount required to keep dust low while also allowing a dry composition to be “free flowing”. A suitable “free flowing” characteristic can be determined by a funnel flow test or free flow test. A dedusting agent can be included in a feed in an amount between about 5-40 lbs/ton.

The one or more essential oils can be selected for their anti-microbial, antioxidant, anti-fungal, anti-viral, and anti-coccidial properties. The one or more essential oils can be selected based on mode of action, which can include one or more of dissipate potassium gradient; permeabilize membrane; damage cell envelope; permeabilize membrane, cytoplasmic leakage, inhibit respiration; perturb membrane permeability, release cellular content; cell lysis and damage cell surface; inhibit respiration and extracellular production; compromise cytoplasmic membrane; cell membrane protein and enzyme function; damage cell wall; membrane-rigidifying effects, and affect lipid polymorphism. For example, in an embodiment, the one or more essential oils can destroy oocysts. In an embodiment, the oocysts can be selected from the genus Eimeria, Isospora, and Cryptosporidia.

### Methods of Preparing the Compositions

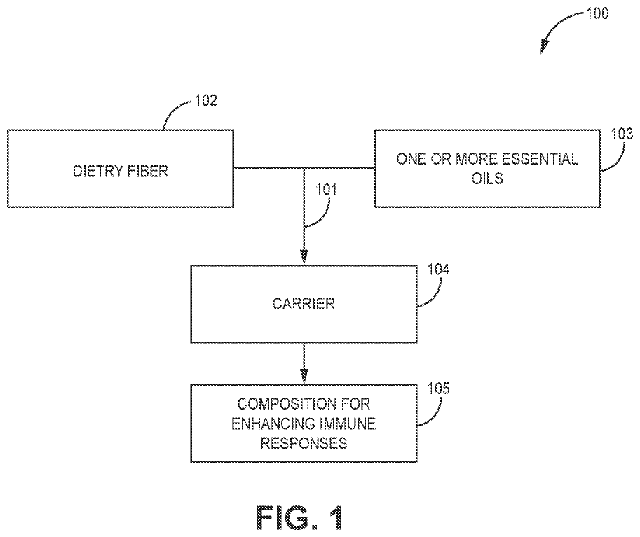

FIG. 1 is a flowchart of a method of preparing a composition, according to one or more embodiments of the present disclosure. As shown in FIG. 1, the method 100 can comprise contacting 101 a dietary fiber 102 and one or more essential oils 103 with a carrier 104 sufficient to form a composition for enhancing immune responses in subjects having or susceptible to having infections and/or diseases caused by pathogens 105. The discussion of the ratio of dietary fibers to essential oils, as well as the amount of dietary fibers, essential oils, and carrier, among other things, provided above is hereby incorporated by reference in its entirety.

The step 101 includes contacting a dietary fiber 102 and one or more essential oils 103 with a carrier 104 to form the composition 105. The contacting 101 can proceed by bringing the dietary fiber 102, one or more essential oils 103, and carrier 104 into physical contact, or close or immediate proximity. The dietary fiber 102, essential oils 103, and carrier 104 can be contacted simultaneously, or sequentially, in any order. In an embodiment, the composition can be prepared by casting the one or more essential oils on a solid carrier, such as animal feed, until the solid carrier sorbs (e.g., absorbs) all or at least a portion of the essential oils, and subsequently adding a dry or wet form of the dietary fiber to the solid carrier. In an embodiment, the composition can be prepared by adding the one or more essential oils and dietary fiber to a liquid carrier, such as reverse osmosis water. The contacting can optionally be sufficient for the dietary fiber to dissolve or solubilize in the liquid carrier. The contacting can also optionally be sufficient to disperse, suspend, and/or emulsify the one or more essential oils in the liquid carrier.

The ratio of dietary fibers to essential oils in the composition can range from about 0:100 to about 100:0, or any increment thereof. In an embodiment, the ratio of dietary fibers to essential oils in the composition can be about 10:1 to about 20:1. For example, the ratio of dietary fibers to essential oils can be about 10:1, about 11:1, about 12:1, about 13:1, about 14:1 about 15:1, about 16:1, about 17:1, about 18:1, about 19:1, about 20:1, or any increment thereof. In one embodiment, the ratio of dietary fibers to essential oils in the composition is about 13:1.

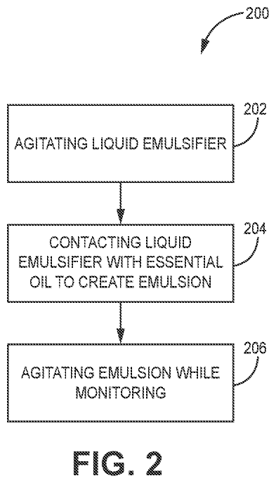

FIG. 2, illustrates a block flow diagram of a method 200 of making an essential oil composition for use in the method 100 discussed above, such as an essential oil emulsification in an aqueous carrier, is shown, according to some embodiments. One or more liquid emulsifiers are agitated 202. The one or more liquid emulsifiers are contacted 204 with one or more essential oils, sufficient to create an emulsion. The emulsion is agitated 206 while monitoring at least an emulsion temperature.

The liquid emulsifier (i.e., water and one or more emulsifiers) is agitated 202 in a vessel, such as by stirring, for a time sufficient to produce visible motion on the surface of the one or more liquid emulsifiers. The visible motion can be from the approximate surface center to one or more surface edges, at the perimeter of the vessel, for example. The time taken to reach such visible motion depends on the type of liquid emulsifier and ratio of emulsifier to water (e.g., viscosity). Once a suitable motion is established at the surface of the liquid emulsifier, one or more essential oils are added (e.g., contacted 204). The agitation of the liquid continues and an emulsion begins to form on contact. The contact rate or addition rate should be slow enough to substantially prevent volatilization of the essential oils.

The agitation continues during the addition of the essential oils. The emulsion begins to form assuming the rate of essential oil addition is slow enough to prevent a high shear environment, adversely affecting the volatilization of the oils. Agitation 206 of the emulsion then continues until the emulsion temperature reaches about 100° F. to about 110° F., about 103° F. to about 108° F. or about 104° F. to about 107° F. As the emulsion forms, the viscosity increases. The method of agitation should be adjusted to compensate for the increase in viscosity. For example, if a stirring method is used, the stirrer or paddle should increase in force to maintain the same level of movement of the liquid as the emulsion thickens.

The final emulsion can have an average droplet size of less than about 25 microns, less than about 15 microns, less than about 10 microns or less than about 5 microns. The smaller droplet size allows for a more stable emulsion and one that previously could not be utilized for animal health or agricultural uses due to instability and high volatilization rates.

### Methods of Administering the Compositions

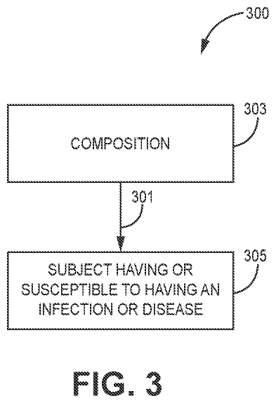

FIG. 3 is a flowchart of a method of administering a composition, according to one or more embodiments of the present disclosure. As shown in FIG. 3, the method 300 can comprise administering 301 a composition 303 to a subject 305 having or susceptible to having an infection and/or disease, wherein the composition comprises a dietary fiber and one or more essential oils.

The step 301 includes administering a composition to a subject having or susceptible to having an infection or disease. The administering 301 is not particularly limited and can include any method suitable for delivering the composition to the subject. The composition can be administered to the subject in solid or liquid form. For example, the composition can be administered as nutritional or feed supplements. In an embodiment, the administering includes mixing the composition with water and/or feed. In an embodiment, the administering includes administering by gavage. In an embodiment, the administering includes bolus feeding. In addition or in the alternative, the administering 301 can include oral ingestion of the composition as a feed or liquid, ingesting the composition in an encapsulated form, or applying the composition topically. Pill-based or encapsulated administrations can be ideal for compositions which are not sufficiently palatable or an animal. However, administration via water or food-based carriers can be preferred for ease of administration. These are provided as examples and thus shall not be limiting. Other methods of administering known in the art can be used herein without departing from the scope of the present disclosure.

The subject can include all manner of animals, including poultry, horses, cows, pigs, and the like. Additionally or alternatively, subject 305 can include humans. Additionally or alternatively, subject 305 can include fish, shrimp, crustaceans, and other aquaculture. Additionally or alternatively, subject 305 can include oviparous and ovuliparous animals.

The compositions can be administered to subjects that have an infection or disease or that are susceptible to having an infection or disease. In an embodiment, an infection can occur upon the introduction of pathogens to the subject and can include the period over or during which the pathogens begin to multiply. In an embodiment, a disease can occur when cells are damaged as a result of, for example, the infection. The subjects to which the compositions are administered can either have an infection or disease, or can be susceptible to an infection or disease. In this way, the compositions can be administered prophylactically or therapeutically, depending on the circumstances of the situation and the needs of the subject, among other things.

The amount of dietary fibers administered to the subject can be selected based on the subject's body weight or mass. In an embodiment, the amount of dietary fiber administered to the subject can range from about 1 mg of dietary fiber per kg of body weight of the subject to about 50 mg of dietary fiber per kg of body weight of the subject, or any increment thereof. For example, the amount of dietary fiber administered to the subject can be about 1 mg of dietary fiber per kg of body weight, about 10 mg of dietary fiber per kg of body weight, about 20 mg of dietary fiber per kg of body weight, about 30 mg of dietary fiber per kg of body weight, about 40 mg of dietary fiber per kg of body weight, or about 50 mg of dietary fiber per kg of body weight, among others. In an embodiment, the amount of dietary fiber administered to the subject can range from about 10 mg of dietary fiber per kg of body to about 20 mg of dietary fiber per kg of body weight.

The amount of essential oils administered to the subject can also be selected based on the subject's body weight or mass. In an embodiment, the total amount of essential oils administered to the subject can range from about 0.1 mg to 1000 mg of essential oil (e.g., pure essential oil, or excluding species other than the essential oil) per kg of body weight of the subject, or any increment thereof. The amount of essential oil per kg of body weight can depend on the essential oil, its minimum inhibitory concentration (MIC), and the pathogen. For example, essential oils, such as cinnamon essential oil, thyme essential oil, and/or oregano essential oil, among others, can exhibit low MICs with respect to a plurality of pathogens. In an embodiment, the amount of essential oil suitable for enhancing immune responses in subjects can range from about 0.5 mg to about 1.0 mg of essential oil per kg of body weight. Other essential oils may require more than 1.0 mg of essential oil per kg of body weight in order to enhance immune responses in subjects.

In an embodiment, the amount of essential oils administered to the subject can depend on the species of the subject, the size of the subject, and the health status of a subject. For example, essential oils can be administered regularly (i.e., daily) as a routine nutritional and health supplement, as an intervention (i.e., for several days or for the duration of a particular episode) in response to or in conjunction with increased stress, disease, birth, or other factors, or as a one-time administration during birth or a severe infection, disease, or injury. In some embodiments, essential oils can be administered 201 to a subject as a routine feed, in a dose of about 0.1 mg of essential oil fraction to about 10 mg of essential oil fraction per kg of subject body weight (mg/kg), about 0.25 mg/kg to about 1.1 mg/kg, about 0.5 mg/kg to about 0.75 mg/kg, or about 0.5 mg/kg. Routine feed can include water, liquid feed, and solid feeds. The essential oils are generally administered in an amount above 5 ppm essential oil relative to the total feed dose, or approximately about 0.01 mg/kg. The dosage amount of essential oils can be varied based on the health of a subject. For example, an amount of essential oil per does can be increased in response to a subject showing a deterioration in health, or other physical characteristic.

In some embodiments, essential oils can be administered to a subject as an intervention in a dose of about 1.0 mg/kg to about 10 mg/kg, about 2 mg/kg to about 7 mg/kg, or about 3.5 mg/kg. In some embodiments, essential oils can be administered to a subject as a one-time administration in a dose of about 10 mg/kg to about 30 mg/kg, about 13 mg/kg of essential oil fraction to about 24 mg/kg, or about 17 mg/kg.

The following Examples are intended to illustrate the above invention and should not be construed as to narrow its scope. One skilled in the art will readily recognize that the Examiners suggest many other ways in which the invention could be practiced. It should be understand that numerous variations and modifications may be made while remaining within the scope of the invention.

### Example 1

The following Example relates to a study that measured the anticoccidial efficacy/sensitivity of all natural feed additives against a mixture of Eimeria acervulina, E. maxima, and E. tenella. The experiment consisted of 42 cages starting with 8 chicks each. The treatments were replicated in 6 blocks, randomized within blocks of 7 cages each. Treatments to randomly assigned to cages. Cages were blocked by location in the battery with block size equal to treatments. The experimental design of treatments is summarized in Table 2 and Treatments/Test Products A-D are summarized in Table 3, both of which are provided below:

TABLE 2
Experimental Design
Experimental Design

| | | Infected/ | Cages/ | Birds/ |
|---|---|---|---|---|
| Trt | Treatment/Dosage | Non- | Trt | Cage |
| 1 | Nonmedicated (NMNI) | NI | 6 | 8 |
| 2 | Nonmedicated (NMI) | I | 6 | 8 |
| 3 | Salinomycin 50 g/t | I | 6 | 8 |
| 4 | Test Product A (2 lbs/US ton) | I | 6 | 8 |
| 5 | Test Product B (2 lbs/US ton) | I | 6 | 8 |
| 6 | Test Product C (2 lbs/US ton) | I | 6 | 8 |
| 7 | Test Product D (2 lbs/US ton) | I | 6 | 8 |

TABLE 3
Treatments A-D Formulations

| | Treatment Formulations | | | |
|---|---|---|---|---|
| | Treatment | Treatment | Treatment | Treatment |
| Ingredient Description | A | B | C | D |
| Inclusion Level in | (@2#/ton) | (@2#/ton) | (@2#/ton) | (@2#/ton) |
| Feed | | | | |
| Limestone | 1500 | 1500 | 1500 | 900 |
| Diatomaceous Earth | 300 | 300 | 300 | 180 |
| Oregano Essential | 200 | 100 | 67 | 40 |
| Oil Concentrate | | | | |
| Thyme Essential | 0 | 100 | 67 | 40 |
| Oil Concentrate | | | | |
| Cinnamaldehyde | 0 | 0 | 66 | 40 |
| Essential | | | | |
| Oil Concentrate | | | | |
| Larch | 0 | 0 | 0 | 800 |
| Arabinogalactan Dry | | | | |
| Total Pounds | 2000 | 2000 | 2000 | 2000 |

### Methods

### Experimental Ration

The appropriate feed treatment commenced at placement Day 0 (day of hatch). An unmedicated commercial type chicken starter compounded with feedstuffs commonly used in the United States was formulated. This ration (in mash form) was fed ad libitum from the date of chick arrival until termination of the study. Experimental treatment feeds were prepared from this basal starter feed. Quantities of all basal feed and test articles used to prepare treatment batches were documented. Treatment diets were mixed to assure a uniform distribution of respective test article. The mixer was flushed to prevent cross contamination. The feed was transferred to a separate building and distributed among cages of the same treatment. Feed was weighed in on Day 0 and non-consumed feed weighed on Days 14 and 20.

### Animals

The study began when the birds were placed (Day 0) at which time they were allocated to the experimental cages. Day of hatch male broiler chicks were obtained from Cobb-Vantress, Cleveland, Ga. The strain was Cobb 500. Breeder flock information was recorded. At the hatchery, the birds were sexed and received routine vaccinations. Only healthy appearing chicks were used in the study. Eight birds were placed per cage. No birds were replaced during the course of the study. Number and disposition of all birds not used for allocation were documented. Birds were weighed on Days 0, 14 and 20.

### Organism Agent

The coccidia inoculum consisted of mixed cultures of three species of Eimeria. The species were E. acervulina, E. maxima, and E. tenella. The histories of the coccidial isolates were included in the source data. The coccidia inoculum was administered by oral gavage to the birds in the infected treatments. The inoculum produced a moderate infection with all species.

### Oocysts Inoculation

Coccidial oocyst inoculation procedures are described in SPFR SOP. On Day 14 of the study each bird in the noninfected treatment received 1 ml of distilled water by oral pipette (p.o.). Birds in the infected treatments received the coccidia inoculum diluted to a 1 ml volume (p.o.).

### Oocysts Per Gram Fecal Material

From all cages, Day 19 to Day 20 samples of the feces passed during this period were collected. Feces collected from each cage were thoroughly mixed and prepared for fecal floatation. Each sample was examined for the number of ooycsts per gram fecal material.

### Lesion Scoring

On Day 20, five birds per cage were humanely euthanized and then lesion scored by cage by Dr. Mathis. The Johnson and Reid, 1970 method of coccidiosis lesion scoring was used to score the infected region(s) of the intestine.

### Management

The facility was checked thoroughly to assure that all cages had water and feed available in each cage. The building temperature was maintained at approximately 80 F. Even, continuous illumination was provided by fluorescent lamps hung vertically along the wall. Feed and water were provided ad libitum. Cages were checked twice daily. Observations including availability of feed, water, temperature and any unusual conditions were recorded. When mortality birds were removed from cages, the cage number, date, weight of the bird, sex and probable cause of death were recorded.

### Data Collected

The following schedule was followed for data collection:

Day 0—Feed issued, birds weighed by cage and allocated.

Day 14—Birds & feed weighed by cage. Feed reissued. Coccidia challenged.

Day 19—Dropping pans cleaned.

Day 20—Birds weighed by cage. Remaining feed weighed. Fecal material collected by cage. Birds coccidia lesion scored.

Death weights recorded with autopsy to determine probable cause of death. Clinical observations, twice daily, were recorded.

### Disposal of Test Animals and Feed

All birds and remaining feed were buried in SPFR pit according to SPFR SOPs. Records of disposal were included in the source data.

### Data Analysis

Mean for group weight gain, feed consumption, feed conversion, lesion scores, OPGs, and mortality were calculated and analyzed by standard statistical procedures.

### DISCUSSION OF RESULTS

### Performance

The results of this study showed that BW (Body Weight) gain (0-20 days) was not significantly different (P≥0.05) for the NMI (Nonmedicated Infected) group and the groups fed Test Products A, B or C. The BW gain for the group fed Test Product D was significantly better than NMI group but significantly lower than the NMNI (Nonmedicated Noninfected) and Salinomycin groups. Adj FCR (Adjusted Feed Conversion) was not significantly different for the NMNI, Salinomycin and Test Product D groups and was significantly better than the NMI group. The Adj FCR was not significantly different for the NMI. Test Product A, B & C groups.

### Oocysts Counts and Coccidia Lesions

As expected, the oocysts count for the NMNI group was zero. The highest oocysts count was seen in the NMI group and while numerically less the oocysts count for Test Product B, C & D was not significantly different. The oocysts count for group on Test Product A was significantly lower than the NMI group but significantly higher than the NMNI and Salinomycin groups. While not significantly different the Salinomycin group averaged 20,457 OPG versus the 0 for the NMNI group.

The lesion score was the highest for the NMI group and was significantly different than all the other treatments. The lesion scores for the NMNI group was 0 and was significantly different than all the other treatments. After the NMNI group the Salinomycin and Test Product D had the lowest lesion scores and were not significantly different from one another. The lesion score for Test Product A, B & C fell in the middle being significantly better than the NMI group but significantly poorer than the Salinomycin and Test Product D.

This trial supports previous trials that have shown that essential oils can have protective effects when birds are experimentally challenged with coccidia, with some essential oils being more effective than others. Test Product A was shown to be the most effective in reducing the number of oocysts being shed. Test Product D a combination of essential oils and dietary fibers while not as effective as Test Product A at reducing oocysts shedding was significantly better at improving Adj FCR and reducing lesion scores. The Adj FCR and lesion scores for Test Product D and Salinomycin were not significantly different. This would indicate that feeding Test Product D should give similar results to feeding Salinomycin a commonly used poultry coccidiostat.

Results Tables

| | | | | | Weight | Weight |
|---|---|---|---|---|---|---|
| | Feed | Feed | Adjusted | Adjusted | Gain | Gain |
| | Intake | Intake | FCR | FCR | (kg) | (kg) |
| Treatment | D 0-20 | D 14-20 | D 0-20 | D 14-20 | D 0-20 | D 14-20 |
| 1. No Additive, No Cocci | 6.677a | 3.015a | 1.705c | 1.424d | 0.497a | 0.265a |
| 2. No Addtiive, Cocci | 6.344ab | 2.660bc | 2.239a | 2.680a | 0.359c | 0.126d |
| 3. Salinomycin 50 g/t | 6.134b | 2.755b | 1.666c | 1.531d | 0.464a | 0.225b |
| 4. Test Product A, 2 lbs/US t | 5.952b | 2.678b | 2.030ab | 2.221bc | 0.381bc | 0.159c |
| 5. Test Product B. 2 lbs/US t | 6.179ab | 2.625bc | 2.235a | 2.391b | 0.356c | 0.139cd |
| 6. Test Product C, 2 lbs/US t | 5.867b | 2.474c | 1.996ab | 2.342b | 0.375bc | 0.138cd |
| 7. Test Product D, 2 lbs/US t | 6.154ab | 2.597bc | 1.705bc | 2.057c | 0.411b | 0.159c |

| Lesion Scores | Eimeria | | | |
|---|---|---|---|---|
| Treatment | acervulina | maxima | tenella | AVG |
| 1. No Additive, No Cocci | 0.0e | 0.0e | 0.0c | 0.0c |
| 2. No Addtiive, Cocci | 1.8a | 1.7a | 1.6a | 1.7a |
| 3. Salinomycin 50 g/t | 0.8d | 0.7d | 0.9b | 0.8d |
| 4. Test Product A, 2 lbs/US t | 1.3bc | 1.1bc | 1.0b | 1.1bc |
| 5. Test Product B. 2 lbs/US t | 1.3b | 1.3b | 1.1b | 1.2b |
| 6. Test Product C. 2 lbs/US t | 1.4b | 1.3b | 1.1b | 1.3b |
| 7. Test Product D, 2 lbs/US t | 1.1c | 1.0cd | 0.7b | 0.9cd |

| Oocysts/Gram | Eimeria | | | |
|---|---|---|---|---|
| Treatment | acervulina | maxima | tenella | Total |
| 1. No Additive, No Cocci | 0d | 0c | 0c | 0c |
| 2. No Addtiive, Cocci | 57475a | 570abc | 9090a | 67134a |
| 3. Salinomycin 50 g/t | 18403cd | 212bc | 1843bc | 26457c |
| 4. Test Product A, 2 lbs/US t | 34393bc | 838ab | 6745ab | 41976b |
| 5. Test Product B. 2 lbs/US t | 52696ab | 1240a | 7861a | 61796ab |
| 6. Test Product C. 2 lbs/US t | 48597ab | 670abc | 8196a | 57464ab |
| 7. Test Product D, 2 lbs/US t | 52316ab | 592abc | 10698a | 63516a |

### Example 2

### 3-Oil Screening Trial

640 1-day-old Cobb 500 cockerels were wing banded, weighed and randomly assigned to pens on the day of delivery. Each pen was 3 feet wide by 2 feet long by 2 feet tall holding 10 birds each; density was 0.6 sq. ft. per bird. The pens were contained within two battery cages in a single barn, each with battery cage having 4 levels and 8 pens per level for a total of 32 pens per battery. The experimental period was 35 days divided into three (3) feeding stages, starter, grower and finisher. Bird weight, average daily gain (ADG), average daily feed intake (ADFI), feed to gain (FG) and Coccidiosis oocyte counts were determined at each period. This study was performed with a large number of treatments in order to screen a number of possible treatments for further future investigations. The treatments included (1) a negative control (no stress, no treatment); (2) positive control, no treatment with stress (bird stress induced by administering ten times the normal level of the Advent coccidiosis vaccine in gel on top of the feed, a typical experimental broiler chick stress); (3) SO1 (75% limestone, 15% diatomaceous earth, 5% Larafeed and 5% essential oil at equal parts oregano essential oil, thyme essential oil, and cinnamaldehyde) at 0.75 pounds per ton of feed plus the cocci stress; (4) Essential Oil Product A (75% limestone, 15% diatomaceous earth, 5% Larafeed and 5% essential oil at 66.6% thyme essential oil, and 33.3% cinnamaldehyde), a combination of thyme and cinnamon oils, with the cocci stress, administered at 0.75 pounds per ton of complete feed; (5) artificial cinnamaldehyde, with the Advent vaccine cocci stress, administered at 0.75 pounds per ton; (6) Essential Oil Product B (60% Essential Oil Product C and 40% Larafeed), administered at 1.33 pounds per ton, with the Advent vaccine cocci stress; (7) Essential Oil Product C, described above, in a step-down program administered at 0.9, 0.6 and 0.4 pounds per ton in the starter, grower and finisher phases, respectively.

Average bird weights for these nine (9) treatments, from lowest to highest at thirty-five (35) days were: (a) Positive control at 1984.0 grams, as might be expected; (b) Negative control at 2039.9 grams; (c) Essential Oil Product B at 2093.1 grams; (d) Essential Oil Product C at 2153.4 grams; (e) Essential Oil Product A at 2172.0 grams; (f) cinnamaldehyde at 2180.2 grams, and (g) SO1 at 2202.4 grams. In this trial the top two treatments in bird weight performance was SOL the 3-oil blend of oregano, thyme and cinnamaldehyde and ActiFibe, the prebiotic fiber. The positive impact of this product on gut health and host immunity led to higher feed efficiency and weight gain of all other products.

The feed-to-gain ratio (F:G) results in this trial were in the range of 1.19 to 1.26 for the starter phase, between 2.89 and 3.66 for the grower phase and 0.98 to 1.09 for the finisher phase, with lower F:G values being preferred. The 3-oil blend SO1 treatment resulted in the lowest F:G value in the starter phase at 1.19, the grower phase at 2.89, and just slightly above the lowest F:G value, 0.98, in the finisher phase at 1.00. Therefore birds that received the SO1 3-oil treatment not only grew the most, but were the most efficient in feed conversion of all the treatments in two of the three phases and next to best in the third phase. More specifically, the combination of the three oils in feed resulted in broiler birds gaining more weight and doing it more efficiently than the other two oil essential oil combinations. The SO1-treated birds also had the lowest mortality of all treatment groups.

### Example 3

### 3-Oil Blend Challenge Against Commercial, Products and Industry Standard Treatments

640 1-day-old Cobb 500 cockerels were wing banded, weighed and randomly assigned to pens on the day of delivery. Each pen was 3 feet wide by 2 feet long by 2 feet tall holding 10 birds each; density was 0.6 sq. ft. per bird. The pens were contained within two battery cages in a single barn, each with battery cage having 4 levels and 8 pens per level for a total of 32 pens per battery. The experimental period was 42 days divided into four feeding stages: starter, grower, finisher and withdrawal. Bird weight, average daily gain (ADG), average daily feed intake (ADFI), feed to gain (FG) and Coccidiosis oocyte counts were determined at each period. This study was conducted with four treatments in order to compare Essential Oil Product C and SO1 in a challenge setting using Advent Coccidiosis vaccine at 10× the normal level, comparing it to a typical industry treatment protocol with Coban/BMD. The treatments were (1) a positive control, no treatment with stress (bird stress induced by administering ten times the normal level of the Advent coccidiosis vaccine in gel on top of the feed, a typical experimental broiler chick stress); (2) SO1 at 1.0 pounds per ton of feed plus the cocci stress; (3) an existing Ralco essential oil blend, Essential Oil Product C, as described above, with the cocci stress, administered at 1.0 pounds per ton of complete feed; and (4) a standard industry treatment protocol, the combination of Coban (Elanco Monensin), a drug used to prevent coccidiosis in chickens, turkey and quail with BMD (Zoetis), a type A Medicated Article for the prevention and control of necrotic enteritis, increased rate of weight gain and improved feed efficiency in poultry, all with the standard cocci vaccine stress with the drugs used at label directions.

Average treatment body weights of the birds at 42 days of age were: 2745.7 grams for the positive control; 2782.6 grams for SO1; 2766.1 grams for Essential Oil Product C, and 2777.8 grams for the Coban/BMD treatment. Feed-to-gain results, respectively, were 2.28, 2.45, 2.17 and 2.47. Hence, SO1 showed the greatest gain of all treatments, including the drug treatment, and further evidenced synergistic effects of the 3-oil blend.

### Example 4

### Vibrio in Shrimp

Several strains of the bacteria Vibrio are responsible for severe economic losses in commercial shrimp production worldwide. Vibrio causes acute hepatopancreas necrosis (AHPND) or early mortality syndrome (EMS). Antibiotics are beginning to lose efficacy against Vibrio and other aquaculture pathogens, in part due to an increased prevalences of resistant bacteria in the human food supply. Essential oils are here demonstrated to be an alternative to antibiotics in this application.

Three Vibrio parahaemolyticus strains isolated from shrimp in Mexico were used to test the efficacy of Essential Oil Product D (RO water 67.75%, Larafeed 12.75%, essential oil 18.5% and 1% xantham gum and sodium alginate, with the essential oil combination at 80:20 oregano oil:thyme oil) and SO1 essential oils to determine their minimum inhibitory concentrations (MIC) or the level at which the essential oils terminate the bacteria. VP 834, 276 and 696 MIC were determined using standard microbiological procedures including serial dilutions. VP 834 was isolated from shrimp stomach in Mexico, VP 276 was isolated from shrimp hepatopancreas in Mexico, and VP 696 was isolated from shrimp larva in Mexico. The results of that testing showed that the MIC of Essential Oil Product D and SO1 against 834, 276 and 696 were, in ppm, (117, 58), (234, 234) and (117, 58), respectively. Given the three strain test results, SO1 was more effective than Essential Oil Product D in two cases, and was equal to Essential Oil Product D in the other. This trial further demonstrates the synergistic benefits of the three oil blend over two oil blends.

### Example 5

A composition was administered through feed to evaluate efficacy towards controlling necrotic enteritis caused by Clostridium perfringens in broiler chickens. An experimental house was divided into pens of equal size, arranged along a central aisle. The birds were kept in 24 pens each having an area of 5×10=50 ft². All pens had approximately 4 inches of built-up litter with a coating of fresh pine shavings. A built up litter was used for poultry production to simulate commercial conditions. This built-up litter also was a source of coccidial oocysts. The initial stocking density, after subtracting out for equipment, was ˜0.83 ft²/bird or 50 birds per pen. Each pen has 5 feet high side walls with bottom 1½ feet being of solid wood to prevent bird migration.

The experiment consisted of 24 pens starting with 50 male broiler chickens per pen. The treatments were replicated in eight blocks, randomized within blocks of eight pens each. A randomization procedure for pen assignment for treatments and blocks was provided by SPFR.

All birds were spray vaccinated with coccidia vaccine (Coccivac-B52) at SPFR with the label recommended dosage on day of hatch. Standard floor pen management practices were used throughout the experiment. The temperature of the building was monitored. Environmental conditions during the trial (temperature) were appropriate to the age of the animals. Illumination was provided by fluorescent bulbs placed above the pens. The lighting scheme was 24 hours of light from day 1 to day 14, then 18 hours of light to day 42.

Broilers in the positive control and cocci treatments were given C. perfringens via water daily from day 19-21 following an Eimeria Maxima challenge at day 14 to induce necrotic enteritis.

This experiment examined the effect of an essential oil blend (hereinafter, referred to as “Cocci Product”) in broilers with clinical necrotic enteritis. Inclusion of the cocci product in broiler feed increased weight gain, decreased mortality, decreased lesion scores, and decreased FCR relative to broilers with necrotic enteritis. Additionally, average body weight at termination and cumulative mortality was significantly similar to uninfected broilers. Significant reduction in lesion scores in infected broilers by administration of the Cocci Product demonstrated improvements in gut health. Additional significant improvements in performance characteristics resulted from this improved gut environment.

### Treatments

| | NE | |
|---|---|---|
| Treatment | Challenge | Product inclusion |
| No Additive, No CP (neg. control) | No | None |
| No Additive, CP (positive control) | Yes | None |
| Cocci Product | Yes | 0.44 lb/ton |

### Results

| Day 21 | Feed | Adj. | Wt. |
|---|---|---|---|
| Treatment | Intake | FCR | Gain (kg) |
| No Additive, No CP | 45.24a | 1.459c | 0.588a |
| No Additive, CP | 43.90a | 1.677a | 0.493b |
| Cocci Product | 42.17b | 1.561b | 0.511b |
| Day 35 | Feed | Adj. | Wt. |
| Treatment | Intake | FCR | Gain (kg) |
| No Additive, No CP | 108.61a | 1.525c | 1.488a |
| No Additive, CP | 108.46a | 1.749a | 1.371b |
| Cocci Product | 109.81a | 1.604b | 1.479a |

| Day 42 | Feed | Adj. | Wt. | Percent |
|---|---|---|---|---|
| Treatment | Intake | FCR | Gain (kg) | Mortality |
| No Additive, No CP | 167.44a | 1.627c | 2.267a | 8.2b |
| No Additive, CP | 161.09b | 1.840a | 2.023b | 15.2a |
| Cocci Product | 169.79a | 1.699b | 2.219a | 8.5b |

| Necrotic Enteritis | Lesion | % NE |
|---|---|---|
| Treatment | Scores | Mortality |
| No Additive, No CP | 0.0c | 0.0c |
| No Additive, CP | 1.4a | 13.0a |
| Cocci Product | 0.4b | 4.3b |

What is claimed is:

1. A composition for enhancing immune responses in poultry, comprising:
a dietary fiber,
one or more essential oils, and
a carrier.

2. The composition of claim 1, wherein the dietary fiber composition is selected from larch arabinogalacatan, polydextrose, chitin, psyllium, methylcellulose, hydrolyzed guar, guar, soy polysaccharide, oat bran, pectin, inulin, fructooligosaccharides (FOS), gum arabic, and combinations thereof.

3. The composition of claim 1, wherein the dietary fiber composition is larch arabinogalactan.

4. The composition of claim 3, wherein the larch arabinogalactan comprises arabinogalactan and polyphenols from larch trees.

5. The composition of claim 3, wherein the arabinogalactan is a highly branched polysaccharide having a molecular weight between about 10 kDa and 60 kDa.

6. The composition of claim 3, wherein the polyphenols are present at a concentration ranging from about 1 wt % to about 4 wt %.

7. The composition of claim 1, wherein the essential oils are selected from oregano essential oil, thyme essential oil, cinnamon bark essential oil, lavender essential oil, bay leaf essential oil, cinnamon leaf essential oil, laurel leaf essential oil, lemon grass essential oil, spearmint essential oil, peppermint essential oil, rosemary essential oil, clary sage essential oil, and combinations thereof.

8. The composition of claim 1, wherein the one or more essential oils include at least one of oregano essential oil, thyme essential oil, and cinnamon bark essential oil.

9. The composition of claim 1, wherein the carrier is selected from water, limestone, diatomaceous earth, and animal feed.

10. The composition of claim 1, wherein the carrier is a liquid carrier in which the dietary fiber is dissolved and the one or more essential oils are dispersed.

11. The composition of claim 1, wherein the carrier is a solid carrier in which the dietary fiber and one or more essential oils are sorbed.

12. The composition of claim 1, wherein a ratio of the dietary fiber composition to the essential oils is between 0:100 and 100:0.

13. A method of administering a composition, comprising:
administering a composition to a subject having or susceptible to having a gastrointestinal infection and/or disease caused by a pathogen, wherein the composition comprises a dietary fiber, one or more essential oils, and a carrier.

14. The method of claim 13, wherein the administering includes mixing the composition with water or feed, administering by gavage, and bolus feeding.

15. The method of claim 13, wherein the composition comprises larch arabinogalactan and one or more of cinnamon essential oil, thyme essential oil, and oregano essential oil.

16. The method of claim 13, wherein the pathogen is selected from Eimeria tenella, Eimeria maxima, Eimeria acervulina, Escherichia E. coli, Salmonella typhimurium, Salmonella choleraesuis, Salmonella pullorum, Pseudomonas aeruginosa, Vibrio parahaemolyticus, Vibrio, Aeromonas, Yersinia entercolitica, Klebsiella pneumoniae, Clostridium perfringens, Staphylococcus aureus, Streptococcus agalactiae, Streptococcus uberis, Listeria monocytogenes, Bacillus cereus, Myobacterium avium, Aspergillus, Penicillium, Candida albicans, Streptococcus suis, Salmonella enteritidis, Campylobacter jejuni, Campylobacter coli, and combinations thereof.

17. A method of preparing a composition, comprising:
contacting a dietary fiber and one or more essential oils with a carrier sufficient to form a composition for enhancing immune responses in subjects having or susceptible to having infections and/or diseases caused by gastrointestinal pathogens.

18. The method of claim 17, wherein the dietary fiber is larch arabinogalactan.

19. The method of claim 17, wherein the contacting proceeds by dissolving the dietary fiber in the carrier and dispersing the one or more essential oils in the carrier.

20. The method of claim 17, wherein the contacting proceeds by the carrier sorbing the dietary fiber and one or more essential oils.
